(19) United States
(12) Patent Application Publication
(10) Pub. No.: US 2020/0230070 A1
(43) Pub. Date: Jul. 23, 2020

(54) NANOPARTICLES, METHODS AND USES THEREOF

(71) Applicant: Memorial Sloan-Kettering Cancer Center, New York, NY (US)

(72) Inventors: Moritz Florian Kircher, Boston, MA (US); Matthew Aaron Wall, Seattle, WA (US); Travis Michael Shaffer, Redwood City, CA (US)

(73) Assignee: Memorial Sloan-Kettering Cencer Center, New York, NY (US)

(21) Appl. No.: 16/632,317

(22) PCT Filed: Jul. 20, 2018
(86) PCT No.: PCT/US2018/042984

§ 371 Date: Jan. 17, 2020

Related U.S. Application Data
(60) Provisional application No. 62/535,675, filed on Jul. 21, 2017.

(51) Int. Cl. A61K 9/51; B82Y 5/00

(52) U.S. Cl. CPC A61K 9/5115; B82Y 5/00; A61K 9/0019; A61K 9/5153; A61K 9/5192

(57) ABSTRACT

The present invention provides nanoparticle compositions in which individual nanoparticles comprise one or more dopant entities, as well as methods of making and using such nanoparticle compositions, and various compositions and/or technologies relating to such nanoparticle compositions, their production and/or their use.

### CROSS-REFERENCE TO RELATED APPLICATION

This application claims the benefit of U.S. Provisional Application No. 62/535,675 filed Jul. 21, 2017, the contents of which is hereby incorporated by reference herein in its entirety.

### BACKGROUND

Nanoparticle systems that can incorporate dopant entities have tremendous potential and are useful in a wide variety of contexts.

### SUMMARY

The present invention provides nanoparticles comprising dopant entities, as well as methods of making and using such nanoparticles, and various compositions and/or technologies relating to such nanoparticles, their production and/or their use. Among other things, the present disclosure identifies the source of at least one problem associated with certain technologies for nanoparticle production and/or use, particularly with respect to nanoparticles containing dopants (e.g., SE(R)RS active agent, PET-active radioisotopes, SPECT-active radioisotopes, MM-active metals, therapeutic radioisotopes, fluorescent agents, etc.). In some embodiments, teachings of the present disclosure are particularly applicable to multilayer nanoparticles containing dopants, their production and/or their use and/or compositions that contain them, as well as production and/or use of such compositions.

In one aspect, the present invention is directed to a method of preparing medical isotope labeled nanoparticles, which comprise steps of (1) providing a reaction mixture comprising or consisting of (a) nanoparticles comprising a metal or metal alloy core, a plurality of capping agent entities associated on the core, an outer silica encapsulant layer, and a plurality of SE(R)RS-active agent dopant entities, (b) medical isotopes, and (c) soft cations, and (2) maintaining the reaction mixture under conditions and for a time sufficient for the medical isotopes to bind with the nanoparticles, thereby forming medical isotope labeled nanoparticles. In some embodiments, the reaction mixture is substantially free of chelator.

In some embodiments, the method further comprises a step of isolating the labeled nanoparticles. In some embodiments, the step of isolating the medical isotope labeled nanoparticles comprises centrifuging the reaction mixture. In some embodiments, the step of isolating the medical isotope labeled nanoparticles comprises filtrating the reaction mixture.

In some embodiments, the method further comprises dispersing the isolated medical isotope labeled nanoparticles in an infusion fluid.

In some embodiments, the conditions comprise heating the reaction mixture to a temperature of equal to or greater than 25° C. In some embodiments, the conditions comprise heating the reaction mixture to a temperature of between 45° C. and 80° C. In some embodiments, the conditions comprise heating the reaction mixture to a temperature of equal to or greater than 95° C. In some embodiments, the time is between 5 and 120 minutes.

In some embodiments, the method further comprises administering the medical isotope labeled nanoparticles to a subject in vivo.

In some embodiments, integrity of the medical isotope labeled nanoparticles is not affected by the labeling procedure.

In some embodiments, a binding between the nanoparticles and the medical isotope is covalent. In some embodiments, a binding between the nanoparticles and the medical isotope is non-covalent. In some embodiments, the binding between the nanoparticles and the medical isotope is via chelate bonds.

In some embodiments, the nanoparticles have a longest dimension between 2-1000 nm.

In another aspect, the present invention is directed to a kit for production of medical isotope labeled nanoparticle agents for imaging or therapeutics, which comprises nanoparticles comprising a metal or metal alloy core, a plurality of capping agent entities associated on the core, an outer silica encapsulant layer, and a plurality of SE(R)RS-active agent dopant entities. In some embodiments, the nanoparticles are characterized in that, when exposed to an elevated temperature, the nanoparticles bind a plurality of medical isotopes in the presence of soft cations.

In some embodiments, the kit further comprises reagents for combining the nanoparticles with the plurality of medical isotopes. In some embodiments, the reagents comprise soft cations.

In some embodiments, the kit further comprises a buffer and/or an infusion fluid.

In some embodiments, the kit further comprises a device for administering the medical isotope labeled nanoparticle agent to a subject. In some embodiments, the kit further comprises the device is a syringe.

In some embodiments, the nanoparticles have a longest dimension between 2-1000 nm.

In some embodiments, the nanoparticles bind the plurality of medical isotopes via covalent bonds. In some embodiments, the nanoparticles bind the plurality of medical isotopes via non-covalent bonds. In some embodiments, the nanoparticles bind the plurality of medical isotopes via chelate bonds.

In another aspect, the present invention is directed to a medical isotope labeled nanoparticle agent, which comprises a nanoparticle comprising a metal or metal alloy core, a plurality of capping agent entities associated on the core, an outer silica encapsulant layer, and a plurality of SE(R)RS-active agent dopant entities, the nanoparticle bound to a medical isotope. In some embodiments, the nanoparticle agent is characterized in that it is stable in vivo for at least 3 hours.

In some embodiments, a specific activity of the nanoparticle is no less than 1, 2, 3, 4, or 5 Ci/μmol.

In some embodiments, the plurality of the SE(R)RS-active agent dopant entities is present at sufficiently high density and in sufficient proximity to a surface of the metal or metal alloy that the particle displays ultrahigh Raman sensitivity.

In some embodiments, the nanoparticles agent is characterized in that it localizes in liver, spleen, tumor, lymph node, inflammation, or, infections.

In some embodiments, the nanoparticle agent is characterized in that it comprises at least one targeting moiety/agent. In some embodiments, the targeting moiety comprises at least one agent selected from the list comprising antibodies, peptides, aptamers, small molecular targeting agent and any combination thereof.

In some embodiments, the nanoparticle agent is characterized in that it comprises at least one click reagent. In some embodiments, the click reagent comprises at least one agent selected from the list comprising alkynes, azides, cyclooctynes (e.g., (sulfo-) dibenzocyclooctynes, (1R,8S,9s)-Bicyclo[6.1.0]non-4-yn-9-yls (BCN), (E)-Cyclooctynes, TCO, etc.), isonitriles, ketones, nitrones, oximes, quadricyclanes, and tetrazines.

In some embodiments, the nanoparticle agent is characterized in that the labeled nanoparticle is used for preclinical research, biomedical imaging, therapy, intraoperative imaging, and/or surgery preparation/planning.

In some embodiments, the nanoparticle agent is characterized in that the nanoparticle is bound to the medical isotope via covalent bonds. In some embodiments, the nanoparticle agent is characterized in that the nanoparticle is bound to the medical isotope via non-covalent bonds. In some embodiments, the nanoparticle agent is characterized in that the nanoparticle is bound to the medical isotope via chelate bonds.

### Definitions

In order for the present disclosure to be more readily understood, certain terms are defined below. Additional definitions for, or clarifications of, the following terms and other terms may be set forth throughout the specification.

In this application, the use of “or” means “and/or” unless stated otherwise. As used in this application, the term “comprise” and variations of the term, such as “comprising” and “comprises,” are used in situations where listed items, elements, or steps are included and others may also be included. As used in this application, the terms “about” and “approximately” are used as equivalents. Any numerals used in this application, whether or not preceded by “about” or “approximately” are meant unless otherwise indicated to cover any normal fluctuations (e.g., standard errors or deviations), as would be appreciated by one of ordinary skill in the relevant art. In certain embodiments, the terms “approximately” or “about” refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Administration: As used herein, the term “administration” refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, and vitreal.

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility of the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are bound to one another.

Bound: as used herein, the term “bound” is intended to describe two or more entities that are physically associated with one another by covalent or non-covalent interaction. In some embodiments, two or more entities are determined to be physically associated with one another when the presence of one correlates with the presence of the other. In some embodiments, two or more entities are determined to be physically associated with one another when the ratio reflecting their relative amounts in a given location is stable over time. In some embodiments, non-covalent interactions are or include chelate bonds, hydrogen bonds, van der Waals interactions, hydrophobic interactions, magnetic interactions, electrostatic interactions, etc., and combinations thereof.

Comparable: The term “comparable”, as used herein, refers to two or more agents, entities, situations, sets of conditions, etc. that may not be identical to one another but that are sufficiently similar to permit comparison therebetween so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

Labeled Nanoparticle: as used herein, the term “labeled nanoparticle” refers to a nanoparticle bound (e.g., via covalent or non-covalent (e.g., chelate) bonds) to a medical isotope as described herein. Without wishing to be bound by any particular theory, practice of the present invention traps medical isotopes within nanoparticles, for example through multivalent interaction with electron donor moieties (e.g., oxygens, sulfurs) within the nanoparticle. According to this hypothesis, such medical isotopes are fairly characterized as being bound (e.g., via covalent or non-covalent (e.g., chelate) bonding) by an electron-donor (e.g., oxygen, sulfur) network within nanoparticles. In some embodiments, labeling of nanoparticles occurs via non-covalent binding. In some embodiments, non-covalent bonding between the medical isotope and the nanoparticles is chelate bonding, accomplished without use of traditional chelating agents. In some embodiments, labeling of nanoparticles occurs via formation of covalent bonds between the medical isotope and the nanoparticles. In some embodiments, a composition that is or comprises a labeled nanoparticle is referred to herein as a “labeled nanoparticle agent”. In some embodiments, such an agent includes only a single species of nanoparticle (typically multiple individual nanoparticles of that species). Alternatively, in some embodiments, a particular agent may be or comprise a plurality of different species of labeled nanoparticle.

Medical Isotope: The term “medical isotope” as used herein refers to a metal, a metal-like or non-metal isotope appropriate for use in medical contexts, including clinical research and preclinical applications. In some embodiments, a medical isotope is or comprises a stable isotope; in some such embodiments, a medical isotope is or comprises a radioactive isotope. To give but a few examples, in some embodiments, a medical isotope is or comprises one or more of a nuclear medicine imaging agent, a positron-emitter, a negatron emitter, an alpha emitter, a gamma emitter, a PET-active radioisotope (e.g., Gallium-68, Zirconium-89), SPECT-active radioisotope (e.g., Technetium-99m), a MRI-active material (e.g., Gadolinium, Manganese), a neutron capturing isotope (e.g., Boron-10, Gold-197), a therapeutic radioisotope (e.g., Bismuth-213, Actinium-225), etc. In some particular embodiments, a medical isotope is or comprises a positron-emitter selected from the list including, but not limited to, Zirconium-89, Gallium-68, and Copper-64. In some particular embodiments, a medical isotope is or comprises a PET-active radioisotope or a nuclear medicine imaging agent selected from the list including, but not limited to, Copper-64, Gallium-68, and Zirconium-89. In some particular embodiments, a medical isotope is or comprises a SPECT-active radioisotope selected from the list including, but not limited to, Technetium-99m, Indium-111, Thallium-201, Gallium-67, Tin-117m, or Lutetium-177. In some particular embodiments, a medical isotope is or comprises a MM-active material selected from the list including, but not limited to, Gadolinium, Manganese, Iron, Dysprosium, Holmium, or Erbium. In some particular embodiments, a medical isotope is or comprises a therapeutic (radioactive or non-radioactive) isotope selected from the list including, but not limited to, Actinium-225, Actinium-227, Americium-241, Arsenic-72, Arsenic-74, Astatine-211, Boron-10, Boron-11, Beryllium-7, Bismuth-212, Bismuth-213, Bromine-77, Carbon-11, Carbon-14, Calcium-48, Cadmium-109, Cerium-139, Cerium-141, Californium-252, Cesium-130, Cesium-131, Cesium-137, Chromium-51, Cobalt-55, Cobalt-57, Cobalt-60, Copper-61, Copper-62, Copper-63, Copper-64, Copper-67, Dysprosium-165, Europium-152, Europium-155, Erbium-169, Fluor-18, Gadolinium-153, Gallium-64, Gallium-65, Gallium-67, Gallium-68, Germanium-66, Germanium-68, Germanium-69, Gold-198, Holmium-166, Indium-111, Indium-111m, Iodine-122, Iodine-123, Iodine-124, Iodine-125, Iodine-131, Iodine-132, Iridium-191m, Iridium-192, Iron-55, Iron-59, Krypton-81m, Lead-203, Lead-212, Lutetium-177, Manganese-51, Molybdenum-99 (progenitor to Technetium-99m), Niobium-95, Nitrogen-13, Oxygen-15, Osmium-191, Osmium-194, Palladium-103, Palladium-109, Phosphorus-32, Phosphorus-33, Plutonium-238, Potassium-42, Radium-223, Radium-226, Rhenium-186, Rhenium-188, Rhodium-105, Rubidium-82, Ruthenium-103, Ruthenium-106, Samarium-145, Samarium-153, Scandium-46, Scandium-47, Selenium-72, Selenium-75, Silicon-28, Sodium-24, Strontium-82 (as progenitor of Rubidium-82), Strontium-85, Strontium-89, Strontium-90, Strontium-92, Sulfur-35, Tantalum-178, Tantalum-179, Tantalum-182, Technetium-96, Technetium-99m, Terbium-149, Thallium-201, Thorium-227, Thorium-228, Thorium-229, Thulium-170, Thulium-171, Tin-117m, Tritium, Tungsten-188, Xenon-127, Xenon-133, Ytterbium-169, Ytterbium-177 (as a progenitor of Lu-177), Yttrium-89, Yttrium-90, Yttrium-91, Zinc-65, Zirconium-89, Zirconium-95.

Stable: The term “stable,” when applied to compositions herein, means that the compositions maintain one or more aspects of their physical structure (e.g., size range and/or distribution of particles) over a period of time. In some embodiments, a stable nanoparticle composition is one for which the average particle size, the maximum particle size, the range of particle sizes, and/or the distribution of particle sizes (i.e., the percentage of particles above a designated size and/or outside a designated range of sizes) is maintained for a period of time under specified conditions. In some embodiments, a stable provided composition is one for which a biologically relevant activity is maintained for a period of time. In some embodiments, the period of time is at least about one hour; in some embodiments the period of time is about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, the period of time is within the range of about one (1) day to about twenty-four (24) months, about two (2) weeks to about twelve (12) months, about two (2) months to about five (5) months, etc. For example, if a population of nanoparticles is subjected to prolonged storage, temperature changes, and/or pH changes, and a majority of the nanoparticles in the composition maintain a diameter within a stated range, the nanoparticle composition is stable. In some embodiments, a stable composition is stable at ambient conditions. In some embodiments, a stable composition is stable under biologic conditions (i.e., 37° C. in phosphate buffered saline).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Traditional Chelating Agent: “traditional chelating agent” as used herein refers to those agents that, prior to the present invention, were utilized in the art to bind metal ions in a chelation complex. Labeled nanoparticles as described and prepared herein do not include such traditional chelating agents. Specifically, traditional chelating agents that, in accordance with some embodiments of the present invention, are not included (e.g., at detectable levels—trace/insignificant amounts may be present from other reagents) in nanoparticles for use as described herein may be selected from acetylacetone; aerobactin; aminoethylethanolamine; aminopolycarboxylic acid; ATMP; BAPTA; BDTH2; benzotriazole; bipyridine; 2,2′-bipyridine; 4,4′-bipyridine; 1,2-Bis(dimethylarsino)benzene; 1,2-Bis(dimethylphosphino)ethane; 1,2-Bis(diphenylphosphino)ethane; catechol; CDTA, chelex 100; citric acid; corrole; crown ether; 18-crown-6; cryptand; 2,2,2-cryptand; cyclen; deferasirox; deferiprone; deferoxamine; dexrazoxane; trans-1,2-diaminocyclohexane; 1,2-diaminopropane, dibenzoylmethane; diethylenetriamine; diglyme; 2,3-dihydroxybenzoic acid; dimercaprol; 2,3-dimercapto-1-propanesulfonic acid; dimercaptosuccinic acid; dimethylglioxime; DIOP; diethylenediamine; DOTA; DTPA, DTPMP; EDDHA; EDDS; EDTMP; EGTA; 1,2-ethanedithiol; ethylenediamine; Ethylenediaminetetraacetic acid, etidronic acid; ferrichrome; fluo-4; fura-2; gluconic acid; glyoxal-bis(mesitylimine); hexafluoroacetylacetone; homocitric acid; hydroxamic siderochelates; iminodiacetic acid; indo-1, metal acetylacetonates; metal dithiolene complex; metallacrown; nitrilotriacetic acid; pendetide; penicillamine; pentetic acid; phanephos; phenanthroline; 0-phenylenediamine, phosphonate; phytochelatin, polyaspartic acid; porphin; porphyrin; 3-pyridylnicotinamide; 4-pyridylnicotinamide; sodium diethyldithiocarbamate; sodium polyaspartate; terpyridine; tetramethylethylenediamine; tetraphenylporphyrin; 1,4,7-triazacyclononane; triethylenetetramine; trisodium citrate; 1,4,7-trithiacyclononane; etc.

### BRIEF DESCRIPTION OF THE DRAWING

The Drawing, which is comprised of at least the following Figures, is for illustration purposes only, not for limitation.

FIG. 1 is a schematic showing chelator-free radiolabeling of SE(R)RS nanoparticles, according to an illustrative embodiment disclosed herein. ⁶⁸Ga³⁺ is obtained from a ⁶⁸Ge/⁶⁸Ga generator via direct elution with HCl, rather than purified elution in KOH. The eluent is neutralized by addition of NH₄OH with the hypothesized net effect that K⁺ cations that catalyze silica dissolution are replaced by NH₄⁺ cations that leave the silica shells intact. The ⁶⁸Ga-labeled PET-SE(R)RS NPs are then easily purified by centrifugation.

FIG. 2 is a schematic of a resulting PET-SE(R)RS nanoparticle, according to an illustrative embodiment of the present disclosure. The PET-SE(R)RS nanoparticle is comprised of a gold nanoparticle core, an adsorbed layer of Raman active molecules (IR-780), and a silica shell with a radionuclide (⁶⁸Ga) embedded throughout.

FIG. 3, Panels A-D, depicts characterization of PET-SE(R)RS particles. FIG. 3, Panel A, is a transmission electron microscopy image of PET-SE(R)RS nanoparticles before radiolabeling with ⁶⁸Ga. FIG. 3, Panel B, is a transmission electron micrograph of PET-SE(R)RS nanoparticles after radiolabeling with ⁶⁸Ga at 70° C. for 45 minutes. FIG. 3, Panel C, is a SE(R)RS spectrum of PET-SE(R)RS nanoparticles after radiolabeling at 70° C. for 45 minutes. The characteristic profile of IR-780 is unchanged and the intensity has not decreased. FIG. 3, Panel D, is a plot of instant thin layer chromatography (iTLC) results of radiolabeled SE(R)RS nanoparticles compared to those of free ⁶⁸Ga. The percentage of ⁶⁸Ga bound to SE(R)RS nanoparticles is determined by integrating the signal at the origin and dividing by the total integrated signal.

FIG. 4, Panels A-C, depicts lymph node (LN) tracking with PET-SE(R)RS nanoparticles. FIG. 4, Panel A, is a PET-CT image 4 h after the ⁶⁸Ga-labeled PET-SE(R)RS nanoparticles were injected around the periphery of an orthotopic 4T1 breast tumor. A lymph node can be clearly visualized away from the injection sites (arrowhead).

FIG. 4, Panel B (top), is a photograph (left) and a SE(R)RS spectrum (right) of PET-SE(R)RS nanoparticles being tracked in vivo with a handheld Raman scanner in the cervical LN. FIG. 4, Panel B (bottom), is a photograph (left) and a SE(R)RS spectrum (right) of PET-SE(R)RS nanoparticles being tracked in vivo with a handheld Raman scanner outside the cervical LN. The cervical LN exhibits the characteristic Raman spectrum of the PET-SE(R)RS nanoparticles (top image and spectrum), which is not present outside of the LN (bottom image and spectrum). Accordingly, a quick handheld scan can be performed to guide location and resection of the LN.

FIG. 4, Panel C (top), is a photograph (left) and a SE(R)RS spectrum (right) of PET-SE(R)RS nanoparticles being tracked with a handheld Raman scanner in the excised cervical LN. FIG. 4, Panel C (bottom), is a photograph (left) and a SE(R)RS spectrum (right) of PET-SE(R)RS nanoparticles being tracked in vivo with a handheld Raman scanner outside the region where the cervical lymph node was resected. After resection, the handheld scanner may be used to see that the SE(R)RS spectrum is only detected in the excised tissue (top image and spectrum), indicating clean margins in the resection bed.

FIG. 5, Panels A-E, depicts pre-operative staging and intraoperative imaging of liver cancer using PET-SE(R)RS nanoparticles. FIG. 5, Panel A, is a PET-CT image of a tumor-bearing mouse. Clear filling defects are visible in the liver (arrows) after injection with PET-SE(R)RS nanoparticles. FIG. 5, Panel B, is an intraoperative white light image of the liver from the mouse imaged in FIG. 5, Panel A. Some of the tumors are visible by naked eye due to their large size and differential color. The location of the tumors matches the filling defects of the PET scan. FIG. 5, Panel C, is a maximum intensity projection (MIP) of the PET imaging data, showing healthy liver (high signal) and filling defects corresponding to tumors. FIG. 5, Panel D, is a SE(R)RS image of the liver after injection with PET-SE(R)RS nanoparticles. SE(R)RS imaging of the liver provides a high-resolution, intraoperative map of normal liver (high SE(R)RS signal) and location and extent of the tumors (signal voids). The correlation between PET signal and SE(R)RS signal indicates that the nanoparticles remain intact and active in vivo. FIG. 5, Panel E, is an overlay of the photograph shown in FIG. 5, Panel B, and the SE(R)RS map of FIG. 5, Panel D, showing that the filling defects in the SE(R)RS signal correspond to cancer.

FIG. 6, Panels A-C, shows PET-MR images of liver cancer obtained using PET-SE(R)RS nanoparticles. A mouse with genetically engineered hepatocellular carcinoma (HCC) was injected with 400 μCi of ⁶⁸Ga-labeled PET-SE(R)RS NPs. After 3 hours, micro-PET-MM was performed, and data analysis and PET-MRI co-registration were completed using VivoQuant™ software (InviCro LLC, Boston, USA). The images shown in FIG. 6, Panels A-C, are axial sections through the upper abdomen. FIG. 6, Panel A, is an axial T1-weighted MR image through the liver, demonstrating a hypointense region (dashed-line circle). FIG. 6, Panel B, is a PET image with a signal void (arrow) corresponding to the location of the HCC. FIG. 6, Panel C, is an MRI-PET overlay of the images shown in FIG. 6, Panels A and B.

FIG. 7 is a TEM image showing degradation of silica shells after non-optimized radiolabeling with ⁶⁸Ga from KOH elution. TEM reveals that the silica shells become extremely porous and unstable after the ⁶⁸Ga radiolabeling procedure that had been optimized for pure silica nanoparticles. These nanoparticles rapidly degrade in serum. Scale bar is 100 nm.

FIG. 8, Panels A and B, depicts the influence of water content on silica nucleation and growth. FIG. 8, Panel A, is TEM images of silica nanoparticles synthesized in the absence of gold nanoparticles. Silica nanoparticles were synthesized by a Stober method in ethanol using (from left to right) 1.5 M, 3.0 M, and 4.5 M water for 1 h at room temperature. FIG. 8, Panel B, is TEM images of silica synthesized in the presence of gold nanoparticles using (from left to right) 3.25 M, 5.0 M, and 7.5 M water. Even though the water content is sufficiently high to homogeneously nucleate silica using (from left to right) 3.25 M, 5.0 M, and 7.5 M water, the catalytic effect of the gold nanoparticle surface with respect to the condensation and aggregation of silica nuclei favors shell formation over silica nanoparticle growth. The syntheses in FIG. 8, Panel B, was only allowed to proceed for 25 min, compared to 1 h for the nanoparticles shown in FIG. 8, Panel A. When the water content is increased above 5.0 M, free silica nanoparticles would form within 25 min. Alternatively, if the 3.25 M or 5.0 M syntheses were allowed to progress for 1 h, a substantial amount of free silica nanoparticles would form. Scale bars are 100 nm in FIG. 8, Panel A, and 50 nm in FIG. 8, Panel B.

FIG. 9, Panel A, presents a standard depiction of thin layer chromatogram results. As can be seen, ⁶⁸Ga stays at the origin after incubation with SE(R)RS nanoparticles (traces labeled “⁶⁸Ga-SERRS NP”), but travels with the solvent front in the absence of SE(R)RS nanoparticles (traces labeled “Free ⁶⁸Ga”). As indicated by their labels, Panels B-D plot percentage of bound radiolabel at different values of pH after radiolabeling under various conditions—i.e., 45 minutes at 70° C. (Panel B), 3 h in EDTA (Panel C), or 3 h in serum (Panel D). In each case, as would be understood in the art, percentage of radioactivity bound to the nanoparticles is estimated by the percentage of signal (integrated counts per minute) at the origin of the iTLC paper (Panels B and C) or contained in the >100 kD fraction (Panel D).

FIG. 10, Panels A-C, depicts the characterization of room temperature radiolabeled PET-SE(R)RS nanoparticles. FIG. 10, Panel A, is a transmission electron microscopy image of PET-SE(R)RS nanoparticles after radiolabeling at 25° C. for 5 minutes. Scale bar is 100 nm in FIG. 10, Panel A. FIG. 10, Panel B, is a SE(R)RS spectrum of the PET-SE(R)RS nanoparticles shown in FIG. 10, Panel A. FIG. 10, Panel C, is an instant thin layer chromatogram of PET-SE(R)RS nanoparticles 5 minutes after addition of ⁶⁸Ga at room temperature. The left-most asterisk represents the origin of the iTLC paper (where the SE(R)RS nanoparticles remain) and the right-most asterisk denotes the solvent front (where free ⁶⁸Ga would appear).

FIG. 11, Panels A and B, shows Cerenkov images of the LN with PET-SE(R)RS nanoparticles. FIG. 11, Panel A, is a Cerenkov image in which the entire area was imaged.

FIG. 11, Panel B, is a Cerenkov image obtained while the injection site was covered.

FIG. 12, Panels A-D, depicts a SE(R)RS map of excised LN and resection bed. FIG. 12, Panel A, is a photograph of the same excised tissue shown in FIG. 3, Panel C. FIG. 12, Panel B, is a SE(R)RS image of the same excised tissue shown in FIG. 12, Panel A. SE(R)RS imaging reveals that the LN is completely contained within the resected specimen. FIG. 12, Panel C, is a photograph of the same resection bed shown in FIG. 3, Panel C. FIG. 12, Panel D, is a SE(R)RS map of the same resection bed shown in FIG. 12, Panel A, showing that no SE(R)RS contrast remained after resection and indicating clean surgical margins.

FIG. 13 is a PET-CT image of wild type mouse 5 minutes after injection with PET-SE(R)RS nanoparticles, showing high activity in the liver.

FIG. 14 is a Cerenkov image of PET-SE(R)RS NPs in healthy RES but not in cancerous tissue.

### DETAILED DESCRIPTION

The following description is for illustration and exemplification of the invention only and is not intended to limit the invention to the specific embodiments described.

Unless defined otherwise, technical and scientific terms have the same meaning as commonly understood by one of ordinary skill in the art to which this invention belongs.

All references cited herein, including patent applications and publications, are incorporated by reference in their entirety.

High-precision intraoperative imaging is necessary to delineate the true extent of tumor borders and identify the presence of multiple cancer foci or micrometastases. Failure to remove these malignant cells is a major reason for local recurrences and metastatic spread [1]. It has been previously demonstrated that the new generations of surface-enhanced resonance Raman scattering (SE(R)RS) nanoparticles enable the visualization of the exact extent of malignant and even premalignant lesions after intravenous injection in many different mouse models, with microscopic accuracy [2-10]. Unlike conventional fluorescent imaging agents, SE(R)RS nanoparticles exhibit exceptionally low limits of detection, resistance to photobleaching, unambiguous spectral signatures, and high multiplexing capabilities [2-4]. Surfactant-free synthesis method of the gold nanoparticle cores should further aid in developing nontoxic SE(R)RS nanoparticles with the potential for clinical translation [11, 12]. In contrast to fluorescence imaging agents [13], the unique spectrum of SE(R)RS nanoparticles does not exist in vivo and its specificity is therefore not affected by autofluorescence. The SE(R)RS spectra serve as molecular fingerprints for optical imaging with very high signal-to-background ratios [7]. After resection under white-light, residual cancer can be visualized with SE(R)RS imaging with tumor deposits as small as 100 μm being detectable, thereby minimizing the risk that cancer is left behind during surgery [4, 9, 14]. The increased precision of imaging the true extent of cancerous spread could markedly reduce the need for unnecessary resection of surrounding healthy tissue. It could also enable surgeries that are presently deemed unfeasible due to the proximity of adjacent crucial structures such as nerves or blood vessels, and allow minimally invasive and robotically assisted surgical approaches in situations where currently open surgical approaches are required. Furthermore, SE(R)RS nanoparticles naturally accumulate in the reticuloendothelial system (RES), which has enabled advances in the intraoperative imaging of cancers involving the liver and lymph nodes [10, 15-17].

Although SE(R)RS imaging has many advantages, it does not allow for preoperative surgical planning. Moreover, the high-resolution SE(R)RS imaging necessary to observe small cancerous deposits limits the amount of tissue that can be imaged in an acceptable time frame during surgical procedures. In principle, these challenges could be overcome by the introduction of a complementary whole-body imaging modality that enables rapid pre-operative scans to serve as a roadmap to localize the macroscopic distribution of the tumors deep within organs. Given the very low injected dose of SE(R)RS nanoparticles (e.g., <100 fmol/g), the most important consideration in a complimentary whole-body imaging modality is the limit of detection. Thus, positron emission tomography (PET) (e.g., a sensitivity in the range of 10¹¹-10¹² M) would represent a complementary imaging modality for SE(R)RS nanoparticles [18].

Prior to the present disclosure, efforts to achieve in vivo imaging of PET-active SE(R)RS nanoparticles (PET-SE(R)RS NPs) for clinical applications were not successful. For example, a previous report of Raman nanoparticle radiolabeling describes the attachment of ⁶⁴Cu to silica via a molecular chelator, but did not demonstrate the serum stability of the radiolabeled probe [19]. ⁶⁴Cu can be attached to silica, albeit weakly with poor serum stability [20]. Without wishing to be bound by any particular theory, the present disclosure proposes that competition for radionuclide binding by the nanoparticle itself may complicate efforts to perform traditional molecular-based chelation and requires further characterization to demonstrate stable radiolabeling.

The present disclosure provides an insight that conventional molecular approach to radionuclide chelation presents several additional difficulties. The coordination chemistry changes significantly for different radionuclides, such that a molecule which chelates one species may fail to chelate many others. Moreover, some isotopes do not currently have established and reliable molecular chelators [21]. Additionally, the nanoparticles may not be stable under the conditions necessary for molecular chelation of radioisotopes, such as high temperatures and low or high pH. Even when a molecular chelator can be incorporated onto a nanoparticle surface, undesired side effects may occur, such as changes to the nanoparticle pharmacokinetics. Furthermore, the molecular chelators can be stripped from the nanoparticle surface in vivo, such that the imaging (e.g., positron emission tomography, single-photon emission tomography, etc.) and biodistribution studies sometimes do not correspond to the true distribution of the nanoparticles [22, 23]. These shortcomings may be overcome eventually by a suitable chelator-free approach to SE(R)RS nanoparticle radiolabeling, but no such method existed prior to the present disclosure.

Chelator-free intrinsic radiolabeling has been demonstrated in various systems, such as iron oxide and metal nanoparticles. Established approaches to intrinsic radiolabeling include direct synthesis from radioactive precursors [24], exploitation of specific trapping effects [25, 26], heterogeneous cation exchange reactions [27], and heat-induced coordination of radioactive metal cations [28, 29]. Recently, it has been shown that silica nanoparticles (e.g., without a gold core and free of molecular chelators) can bind ⁸⁹Zr, ⁶⁸Ga, ⁹⁰Y, ¹¹¹I, ¹⁷⁷Lu, and ⁶⁴Cu with stability proportional to the oxophilicity of the radiometal ion [30]. Subsequently, the addition of sulfur to silica nanoparticle surfaces may allow stable radiolabeling of softer, more thiophilic radiometal ions such as ⁶⁴Cu [20]. Chelator-free radiolabeling has also been demonstrated with mesoporous silica nanoparticles by others [31].

The short-lived PET tracer ⁶⁸Ga (t_{1/2}=68 min) may be one of the ideal candidates for the radiolabeling of SE(R)RS nanoprobes, due to i) its oxophilicity, ii) the fact that it is readily available from commercial generators, and iii) its relatively low radiation dose to healthy tissue resulting from its short half-life [30, 32]. The consideration of radiation dose to healthy tissue is particularly important for nanoparticle imaging agents because nanoparticle preparations are sequestered to a significant degree by the RES [33]. Because of the short circulation time of these nanoprobes, ⁶⁸Ga may be great for imaging at the relevant pharmacokinetic time points (e.g., out to 3 hours). The 68-minute half-life provides that ⁶⁸Ga is sufficiently decayed over the course of eight hours to allow SE(R)RS imaging intraoperatively without the potential issue of exposure to radioactivity. However, the ideal radionuclide may vary depending upon the application, and that consideration of the half-life, mechanism of decay (e.g., positron emission necessary for PET), expected dose to healthy or diseased tissue, and coordination chemistry is necessary.

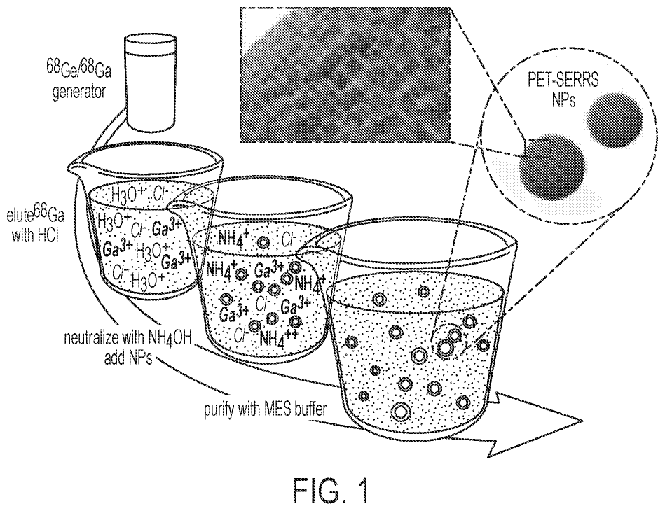

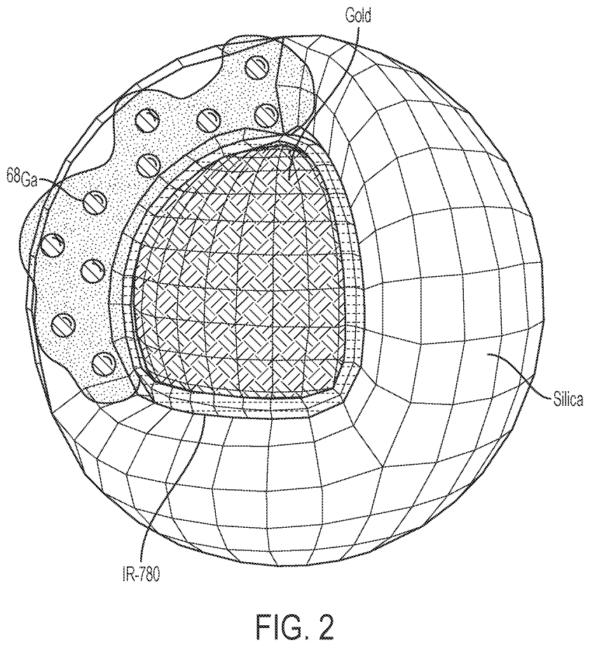

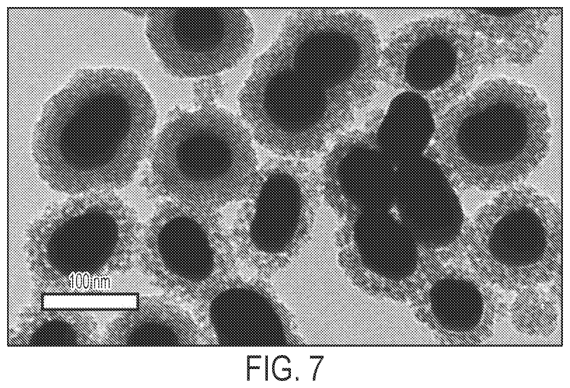

First it was attempted to radiolabel SE(R)RS nanoparticles with ⁶⁸Ga by directly applying the protocol established using pure silica nanoparticles [30]. The SE(R)RS nanoparticles consist of a gold core of ˜60 nm diameter, which is coated with a Raman reporter dye and a ˜30 nm thick silica shell [3, 4]. It was hypothesized that exposure to ⁶⁸Ga under the proper reaction conditions would generate intrinsically radiolabeled SE(R)RS nanoparticles with ⁶⁸Ga distributed throughout the silica shell (FIGS. 1 and 2). A purified elution of ⁶⁸Ga from the ⁶⁸Ge/⁶⁸Ga generator with 0.2 N HCl was performed followed by ⁶⁸Ga trapping on a cartridge, which was then eluted after washing using 0.5 M potassium hydroxide (KOH). This procedure was followed by neutralization with hydrochloric acid (HCl) or glacial acetic acid to achieve pH=7.0-7.5 [34]. In contrast to previous work with pure silica nanoparticles, the silica shell of SE(R)RS nanoparticles were porous and sometimes even disintegrated entirely for some nanoparticles upon exposure to the ⁶⁸Ga solution (FIG. 7). The radiolabeling was unsuccessful.

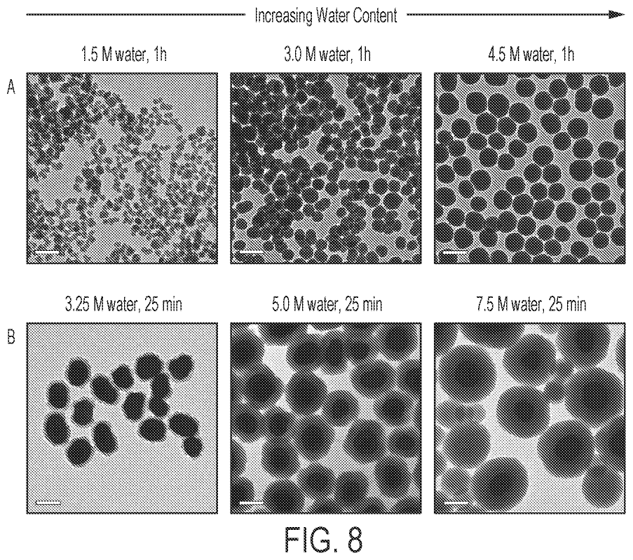

Without wishing to be bound by any particular theory, the decreased stability of the SE(R)RS nanoparticle silica shell compared to a pure silica nanoparticle may be a consequence of their different synthetic conditions. In order to selectively generate silica shells around gold nanoparticles, the homogeneous nucleation of silica is disfavored such that heterogeneous nucleation and growth (e.g., shell formation) occurs, but formation of free silica nanoparticles is minimized. This is achieved by decreasing the rate of hydrolysis and condensation of silica precursors, for example, by decreasing the water concentration during synthesis [35]. Decreased hydrolysis rates may lead to more Si—O—Si broken bonds in the early stages of silica oligomerization and densification because the ethoxy groups of tetraethyl orthosilicate (TEOS) are not completely hydrolyzed [36-38]. Homogenous nucleation is slow, but the gold nanoparticles may provide surfaces to catalyze the condensation and aggregation reactions at the beginning of silica formation, thus enabling preferential nucleation (FIG. 8, Panels A and B). However, the incomplete hydrolysis of silica precursors can lead to broken Si—O—Si bonds within the amorphous silica structure and greater susceptibility to degradation [37].

The present disclosure provides an insight that direct elution of the generator with 0.1 N HCl, and neutralization of the eluent with ammonium hydroxide (NH₄OH), rather than elution of the ⁶⁸Ga generator followed by ⁶⁸Ga trapping on a cartridge and subsequent elution with KOH (FIG. 1), provides improved results. Without wishing to be bound by any particular theory, the present disclosure proposes that, because the NH₄⁺ cations are softer and bulkier than K⁺, they may not intercalate into the silica matrix as well; additionally, the ionic strength of the ⁶⁸Ga solution is decreased using this strategy. Thus, the present disclosure documents that useful improvements can be achieved through use of relatively (with respect to K+) soft and/or bulky cations.

SE(R)RS nanoparticles (10 nM nanoparticle concentration, 3.52×10⁹ g/mol molar mass, 100 μL, pH=8.5) produced as described herein were incubated with the ⁶⁸Ga solution (100 μL, 37 MBq (1.0 mCi), pH=7.0-7.5) for 45 minutes at 70° C., then characterized by instant thin layer chromatography (iTLC), size exclusion (SE) filtration, transmission electron microscopy (TEM), dynamic light scattering, and zeta potential analysis.

Radiolabeling was tested at pH=7.4 as well as pH=8.5 and compared to a free ⁶⁸Ga control that contained no nanoparticles. The radiochemical yield (non-decay corrected) was 90.92+/−1.56% and 95.14+/−3.43% for pH=7.4 and pH=8.5, respectively, while the molar activity was 20-100 Ci/μmol of NPs. Radiochemical yield was calculated as the amount of radioactivity bound to the NP after purification via centrifugal pelleting over the total radioactivity (supernatant plus NP radioactivity). These controls are necessary, as macroscale gallium solutions at neutral pH may result in colloid formation; however, radiochemical (e.g., <nanomolar) concentrations of gallium in buffer precludes colloid formation [40].

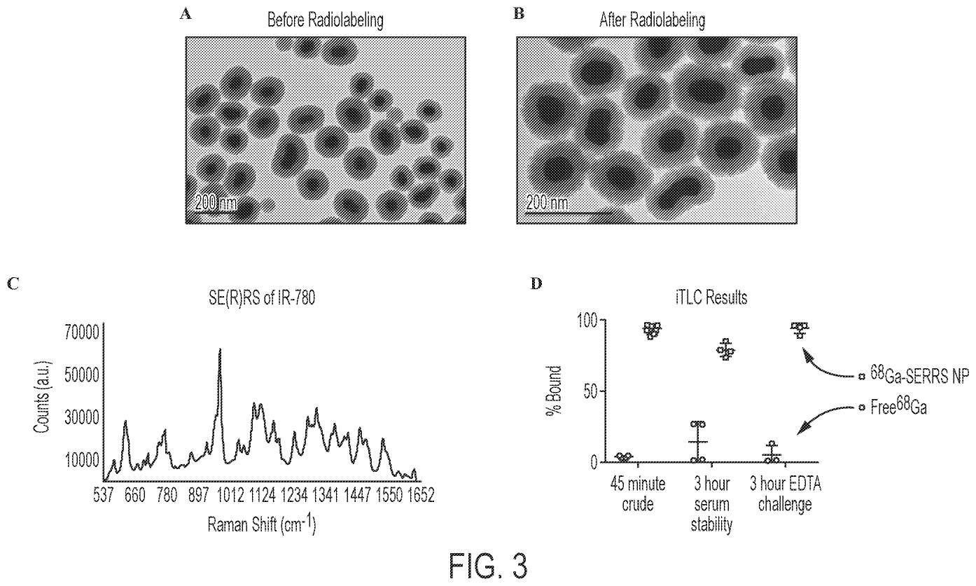

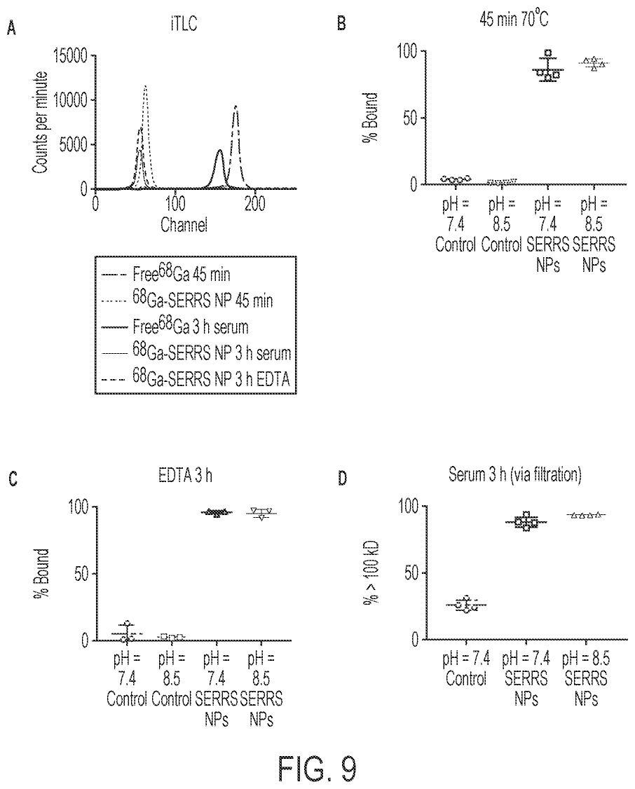

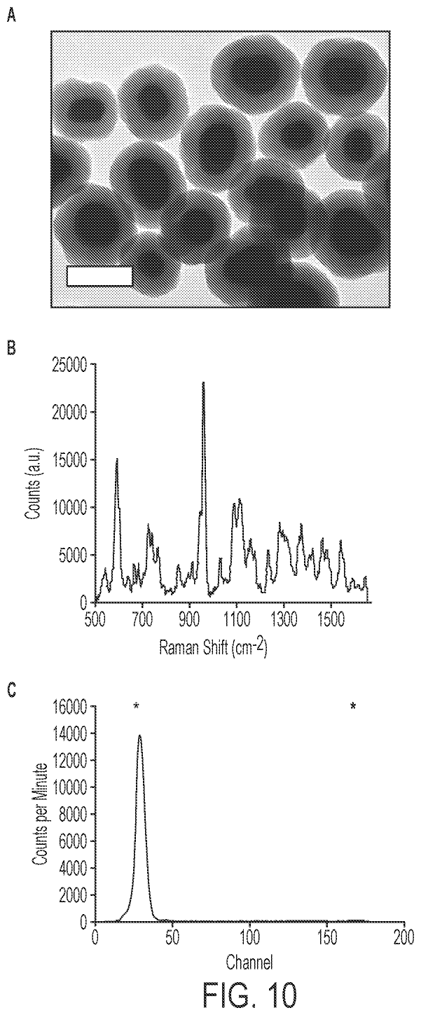

Comparison of TEM images before and after radiolabeling revealed that the stability of the silica shells was improved compared to the prior radiolabeling procedures which included the presence of K⁺ ions (FIG. 3, Panels A-B vs. FIG. 7). However, the porosity of the silica increased and the shell thickness decreased by approximately 7 nm according to dynamic light scattering (Tables 1, 2). The intensity of the SE(R)RS spectrum did not decrease and the ⁶⁸Ga radioactivity remained associated with the nanoparticles (FIG. 3, Panels C-D; FIG. 9, Panels A-D). Successful radiolabeling with ⁶⁸Ga was achieved at 25° C. after only 5 minutes of incubation with SE(R)RS nanoparticles (FIG. 10, Panels A-C).

TABLE 1
Nanoparticle characterization before and after radiolabeling at pH = 7.4.

| | | | |
|---|---|---|---|
| Sample | Z-average (nm) | PDI | Zeta potential (mV) |

IR-780 gold nanoparticle pre-radiolabeled data

| | | | |
|---|---|---|---|
| SE(R)RS NP at pH = 7.4 | 146.8, 145.7, 147.4 | 0.139, 0.128, 0.128 | −35.5, −36.1, −35.1 |
| Mean +/− standard deviation | 146.6 +/− 0.9 | 0.132 +/− 0.006 | −35.6 +/− 0.5 |

IR-780 gold nanoparticle post-radiolabeled data

| | | | |
|---|---|---|---|
| SE(R)RS NP at pH = 7.4 | 131.5, 128.9, 131.6, | 0.221, 0.225, 0.236, | −32.9, −33.6, −32.6, |
| | 134.6, 130.6, 131.6 | 0.231, 0.222, 0.242 | −34.5, −33.2, −33.9 |
| Mean +/− standard deviation | 131.5 +/− 1.9 | 0.230 +/− 0.006 | −33.5 +/− 0.7 |

TABLE 2
Nanoparticle characterization before and after radiolabeling at pH = 8.5.

| | | | |
|---|---|---|---|
| Sample | Z-average (nm) | PDI | Zeta potential (mV) |

IR-780 gold nanoparticle pre-radiolabeled data

| | | | |
|---|---|---|---|
| SE(R)RS NP at pH = 8.5 | 151.4, 148.5, 150.5 | 0.093, 0.110, 0.091 | −46.8, −40.9, −41.0 |
| Mean +/− standard deviation | 150.1 +/− 1.5 | 0.098 +/− 0.010 | −42.9 +/− 3.4 |

IR-780 gold nanoparticle post-radiolabeled data

| | | | |
|---|---|---|---|
| SE(R)RS NP at pH = 8.5 | 123.8, 126.5, 124.9, | 0.248, 0.216, 0.209, | −34.6, −33.8, −32.4, |
| | 137.3, 141.4, 138.7 | 0.231, 0.261, 0.244 | −30.3, −29.8, −29.1 |
| Mean +/− standard deviation | 132.1 +/− 7.9 | 0.235 +/− 0.020 | −31.7 +/− 2.3 |

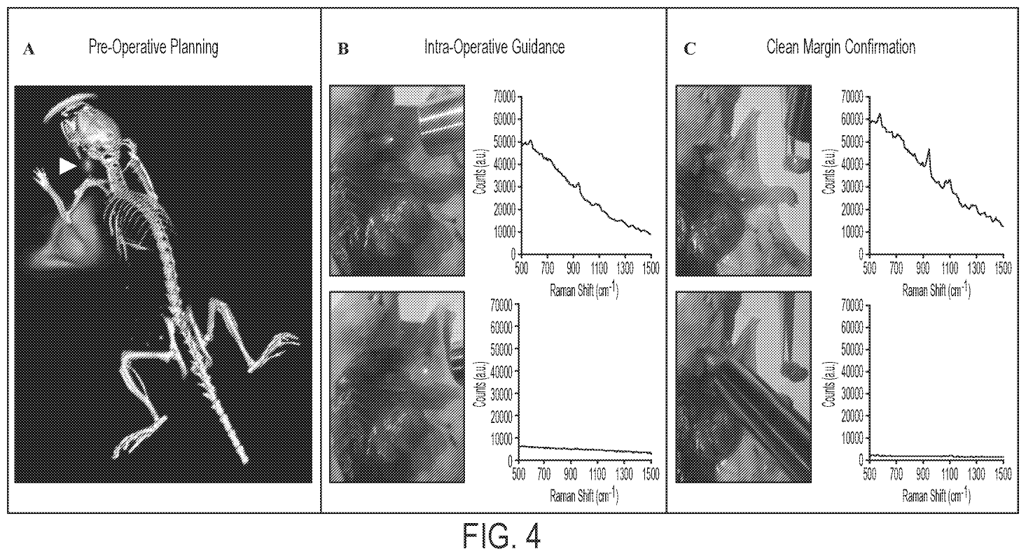

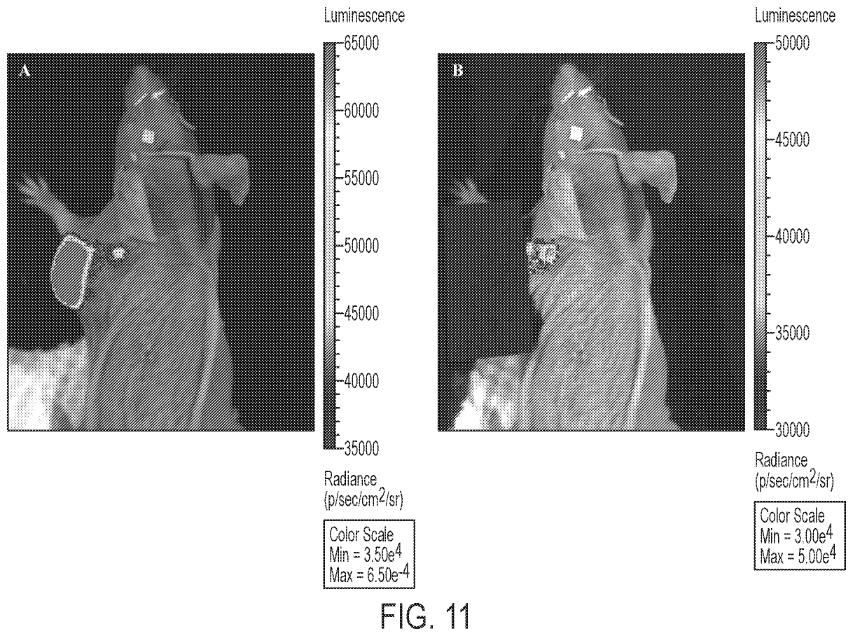

The PET-SE(R)RS NPs were evaluated in vivo by lymph node imaging near the periphery of an orthotopic 4T1 breast cancer tumor. Lymph node imaging may be important for the identification of sentinel lymph nodes, which are routinely excised and examined by pathology in clinical practice to determine if lymphatic metastases exist [10]. Because the location of the primary draining lymphatic vessel may not be determined by visual inspection, sentinel lymph node imaging is performed clinically by injection of a contrast agent in and around a tumor [41]. Previously, it was shown that both radiolabeled silica nanoparticles and SE(R)RS NPs can identify sentinel lymph nodes separately [30, 42], but the combined pre- and intra-operative imaging with a single PET-SE(R)RS imaging agent had not been demonstrated prior to the present disclosure. The PET-SE(R)RS NPs were injected subcutaneously at the tumor periphery and into the tumor itself. PET imaging 4 h post-injection revealed that much of the signal remained concentrated near the tumor, suggesting that most of the PET-SE(R)RS nanoparticles had not migrated from the injection site. The cervical lymph node can be visualized using both PET and Cerenkov imaging with strong contrast at the 4 h time point (FIG. 4, Panel A; FIG. 11, Panels A-B). As Cerenkov luminescence (CL) intensity correlates with the velocity of the charged particle, the high positron energy of ⁶⁸Ga (βₘₐₓ=1.9 MeV) results in greater CL compared to radionuclides such as ¹⁸F [43, 44]. Although the axillary LN is the sentinel node for murine breast tissue, the size and location of the implanted tumor obscures the axillary LN in the small anatomical dimensions of a mouse. The cervical LN drains multiple regions, including the upper extremities. The accumulation of PET-SE(R)RS nanoparticles in the cervical LN occurs because one or more peripheral injection sites falls into its draining pathway [45, 46]. The cervical LN imaging illustrates that LN tracking can be achieved in vivo with PET-SE(R)RS NPs.

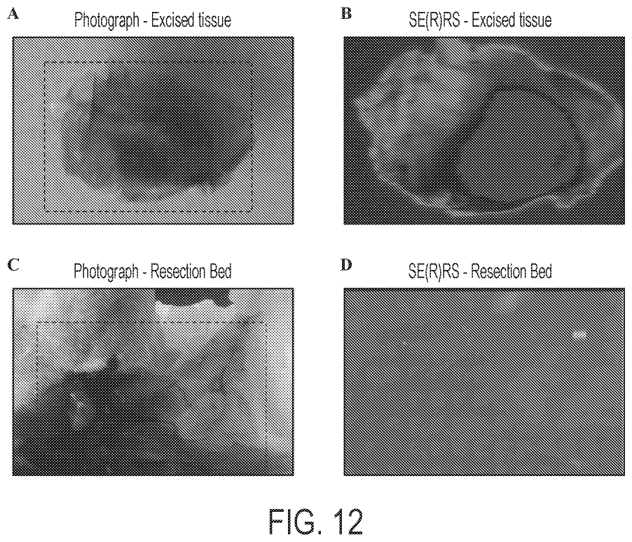

Intraoperative imaging of the cervical LN showed the presence of PET-SE(R)RS NPs via Raman spectroscopy. The characteristic Raman spectrum of the PET-SE(R)RS nanoparticles is detectable with a handheld Raman scanner (FIG. 4, Panels B-C) [6], and allows near real-time analysis of the presence of PET-SE(R)RS NPs. Using the handheld scanner, the presence of the PET-SE(R)RS NP fingerprint spectrum was identified in the regions that also exhibited PET contrast. The PET-SE(R)RS nanoparticles remained intact after subcutaneous injection and migration through the lymphatic channels. The handheld Raman scanner was used to guide surgical resection of the cervical LN, by locating it in vivo, and by confirming that all SE(R)RS-positive tissue had been removed. Post-operative SE(R)RS imaging was performed with the Raman imaging system to corroborate the handheld scanner results, and indeed showed that the lymph node had been completely resected (FIG. 12).

Because the PET-SE(R)RS nanoparticles naturally accumulate in the RES, they should be well suited for imaging cancers of the liver. In particular, the high uptake of nanoparticles in healthy RES tissue and comparatively much lower uptake of nanoparticles in cancerous tissue may delineate tumors in vivo [47, 48]. Because the cancerous regions should contain fewer PET-SE(R)RS NPs than the surrounding liver tissue, the presence of cancer may manifest in filling defects (e.g., regions of little to no signal, surrounded by regions of high signal) with both PET and SE(R)RS imaging. A first proof-of-principle of this concept for non-radiolabeled SE(R)RS nanoparticles was recently shown [49].

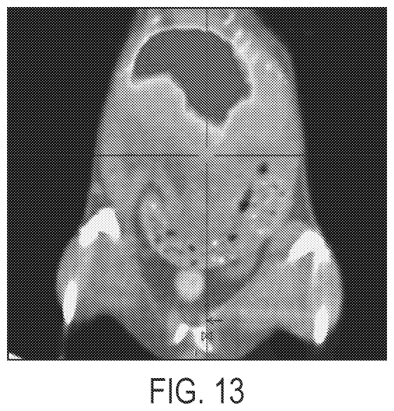

In order to test whether or not the ⁶⁸Ga would remain bound to the SE(R)RS NPs in vivo after intravenous injection, PET-SE(R)RS NPs (150 μL, 10 nM nanoparticles, 500 μCi, 18.5 MBq ⁶⁸Ga) was injected to a wild type mouse and followed the distribution of PET signal on positron emission tomography-computed tomography (PET-CT). After only 5 minutes, the PET signal was already localized to the liver according to PET-CT (FIG. 13). This concentration of PET signal in the liver is consistent with the observed biodistribution of SE(R)RS nanoparticles and with the biodistribution of ⁶⁸Ga-labeled silica nanoparticles [4, 30]. Moreover, this signal distribution is not consistent with the biodistribution of free ⁶⁸Ga (e.g., not bound by a chelator), which shows high blood and bladder signal and relatively low RES signal at 1 and 3 hours post-injection [30]. Thus, the PET-CT results indicate that the ⁶⁸Ga remains sufficiently well bound to the SE(R)RS nanoparticles in vivo.

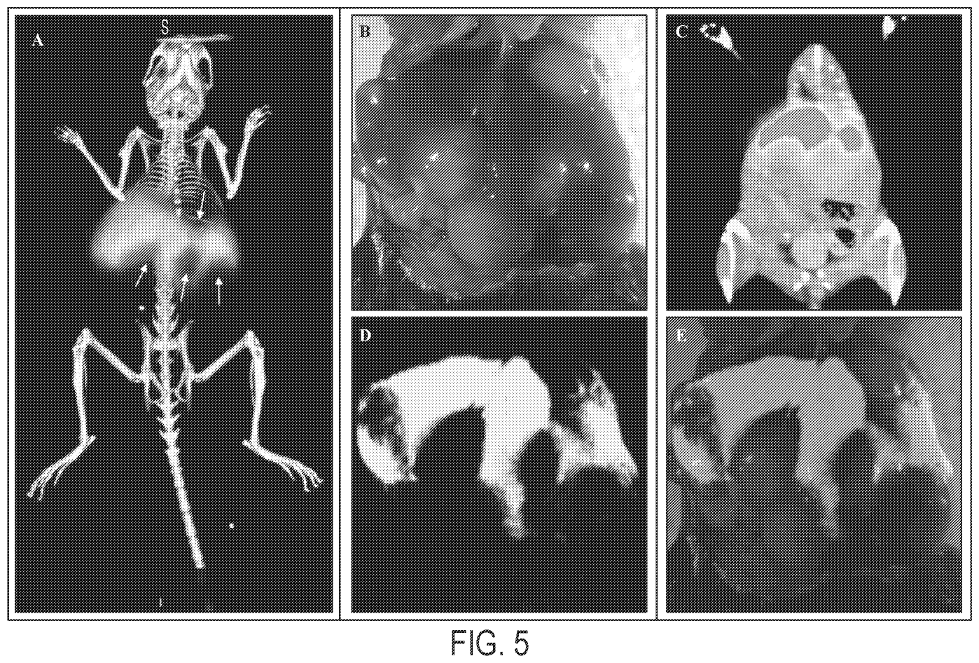

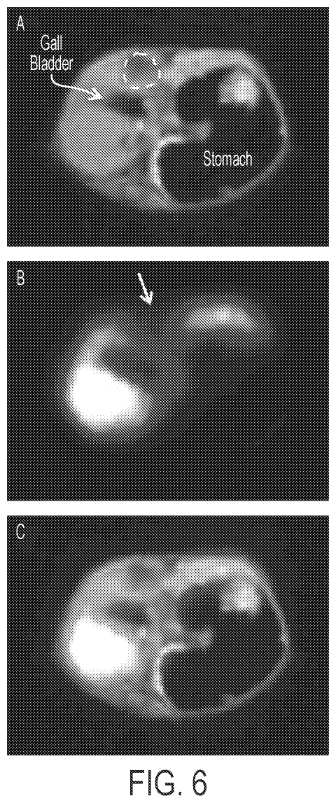

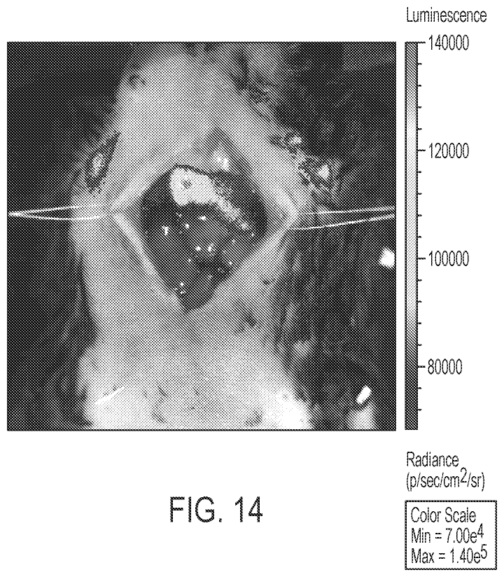

To evaluate the utility of PET-SE(R)RS NPs for delineating liver cancers, the nanoparticles were injected into a mouse that had been genetically engineered to develop hepatocellular carcinomas (HCC) [49]. PET-SE(R)RS NPs (150 μL, 10 nM nanoparticles, 500 μCi, 18.5 MBq ⁶⁸Ga) were intravenously injected into the tail vein and PET and Cerenkov scans were obtained 3 h post-injection (FIG. 5, Panel A; FIG. 14). The PET signal exhibited several distinct filling defects throughout the liver, suggesting the presence of tumors. The livers of the cancer-bearing mouse were exposed surgically and high-resolution SE(R)RS scans were performed in a simulated intraoperative setting. Even without SE(R)RS contrast, some large tumors with sizes and locations corresponding to the filling defects on the PET scan were clearly visible. The SE(R)RS map demonstrated pronounced filling defects where tumors were present, and correlated precisely with the pre-operative PET images (FIG. 5, Panels B-E). The co-registration of PET and SE(R)RS signals in the liver indicate that the PET-SE(R)RS nanoparticles remain intact after intravenous injection and circulation. To ensure that the PET signal corresponded with the healthy tissue throughout the volume of the liver, PET-MRI scans were also performed. The filling defects (e.g., negative contrast) observed via PET matched abnormal signal caused by the tumors on MRI. The PET-SE(R)RS NPs may delineate healthy versus cancerous tissue throughout the liver (FIG. 6, Panels A-C).

The PET-SE(R)RS NPs enable whole-body imaging as a pre-operative roadmap and intraoperative rapid hand-held SE(R)RS scanning or high-resolution SE(R)RS imaging for precise surgical guidance. This invention can work with a variety of other radionuclides, as observed for pure silica nanoparticles. Additionally, this invention introduces a general method for chelator-free radiolabeling of silica-encapsulated materials, thus opening many new avenues for their use in biomedical and other fields.

Materials and Methods

SE(R)RS Nanoparticle Synthesis:

Gold nanoparticles were synthesized by adding 7.5 mL 1% (w/v) sodium citrate to 1.000 L boiling 0.25 mM HAuCl₄. After the nanoparticle dispersion reaches the red color indicative of a substantially complete reaction, it is left to cool for 30 minutes, then concentrated by centrifugation (10 min, 7500×g, 4° C.) and dialyzed overnight (3.5 kDa MWCO; 5 L 18.2 MΩ·cm). The dialyzed gold nanoparticles (140 μL; 2.0 nM) were added to 1 mL absolute ethanol in the presence of 50 μL tetraethoxyorthosilicate (Sigma Aldrich, 99.999%), 20 μL 28% (v/v) ammonium hydroxide (Sigma Aldrich) and 2 μL IR-780 dissolved in N,N-dimethylformamide. IR-780 was selected because of its resonance with the 785 nm laser line, cationic charge, compatibility with silication, and consistency with our previous studies. After 25 minutes of shaking (375 rpm) at ambient conditions in a plastic container, the SE(R)RS-NPs were centrifuged, washed three times with ethanol, and redispersed in water to yield 5 nM SE(R)RS-Nanoprobes.

SE(R)RS Nanoparticle Characterization:

Nanoparticles were imaged by transmission electron microscopy (TEM) acquired on carbon grids (Ted Pella, Inc.) using a JEOL 1200 EX microscope (Peabody, Mass.). Dispersion concentrations were determined by Nanoparticle Tracking Analysis (NTA; Nanosight, Duxbury, Mass.).

Radiolabeling Protocol:

⁶⁸ Ga (t_{1/2}=68 m) was eluted from a ⁶⁸Ge-⁶⁸Ga generator (ANSTO, Australia) as previously described [34], with 555-740 MBq (15-20 mCi) radioactivity per elution. ⁶⁸Ga was eluted with 0.1 N HCl (1.5 mL), and either immediately used or trapped on a filter, washed, and eluted with 0.5 M KOH (0.500 mL). The ⁶⁸Ga HCl solution was neutralized with 28% NH₄OH (13 uL) while the ⁶⁸Ga hydroxide solution was neutralized with concentrated HCl (20-30 μL). Upon neutralization, 50-100 μL of 37 MBq (0.5-1.0 mCi)⁶⁸Ga was immediately added to SE(R)RS nanoparticle dispersions (10 nM, in 100 μL of 10 mM buffer, pH=7.4 or pH=8.5) and incubated at 70° C. on a thermomixer at 500 rpm for 45 minutes. Purification was completed by centrifugation at 10,000 rpm for 120 seconds, followed by decanting the supernatant and redispersing in buffer (e.g., 2-(N-morpholino) ethanesulfonic acid).

In Vivo Experiments:

All animal experiments were done in accordance with protocols approved by the Institutional Animal Care and Use Committee of Memorial Sloan Kettering Cancer Center and followed National Institutes of Health guidelines for animal welfare.

Genetically Engineered Hepatocellular Carcinoma (HCC) Mouse Model:

To generate endogenous HCCs in mice, a sterile 0.9% NaCl solution/plasmid mix was prepared containing 5 μg of DNA for the pT3 EF1a-Myc Sleeping-beauty transposon plasmid mixed with CMV-SB13 Sleeping-beauty transposase plasmid (1:5 ratio) for each injection. A total volume of the plasmid mix corresponding to 10% of body weight was injected into the lateral tail vein of eight to ten week old female FVBN mice (Jackson laboratory, Ben Arbor, USA) in 5-7 s. The pT3 transposon vector was a kind gift by Dr. Xin Chen (UCSF). Approximately 7 weeks after injection, numerous tumors were observed in the livers. Pathological examination showed that the tumors represented poorly differentiated HCCs.

Pet/Ct Imaging:

At predetermined time points (1 h, 3 h) animals were anesthetized with isoflurane (Baxter Healthcare, Deerfield, Ill.) and oxygen gas mixture (2% for induction, 1% for maintenance) and scans were then performed using an Inveon™ PET/CT scanner (Siemens Healthcare Global). Whole body PET static scans were performed recording a minimum of 50 million coincident events, with durations of 10-30 min. The energy and coincidence timing windows were 350-750 keV and 6 ns, respectively. The image data were normalized to correct for non-uniformity of response of the PET, dead-time count losses, positron branching ratio, and physical decay to the time of injection, but no attenuation, scatter, or partial-volume averaging correction was applied. Images were analyzed using ASIPro VM™ software (Concorde Micro-systems). Whole body standard low magnification CT scans were performed with the X-ray tube setup at a voltage of 80 keV and current of 500 μA. The CT scan was acquired using 120 rotational steps for a total of 220 degrees yielding an estimated scan time of 120 s with an exposure of 145 ms per frame.

Cerenkov Luminescence Imaging:

Mice were anesthetized as described previously. Open filters were used for optical scans. 120-300 s scans were completed, depending on the photon flux.

Handheld SE(R)RS Detection:

All handheld Raman measurements were performed using the MiniRam™ Raman handheld scanner (B&W TEK, Inc., Newark, Del.) equipped with a 785 nm laser [6, 10]. Raman spectra were collected with an acquisition time of 1 s and analyzed with B&WSpec 4.01.26 Software (B&W TEK).

Fixed-Microscope SE(R)RS Imaging:

All fixed Raman scans were performed using a Renishaw inVia™ Raman microscope with a 300 mW 785 nm diode laser and 1-inch charge-coupled device detector (1.07 cm⁻¹ spectral resolution). The SE(R)RS spectra were collected with a 5× objective (Leica) and the laser output measured at the objective was 100 mW at 100% laser power. Scans were typically performed at 100 mW laser power, 1.5-s acquisition time, using the StreamLine high-speed acquisition mode. The Raman maps were generated by means of a DCLS algorithm (WiRE™ 3.4 software, Renishaw).

PET-MRI:

A mouse with genetically engineered hepatocellular carcinoma (HCC) was injected with 400 μCi of ⁶⁸Ga-labeled PET-SE(R)RS NPs. After 3 hours, micro-PET-MM was performed (T1-weighted) on a nanoScan PET/MRI system (Mediso USA, Boston, Mass.) and the data analysis and PET-MRI co-registration were completed using VivoQuant™ software (InviCro LLC, Boston, USA).

Radiochemical Yield:

1 μL samples were taken for radioactive instant thin layer chromatography (iTLC) at various time points over the course of 1 hour using silica-gel impregnated iTLC paper (Varian) and analyzed with a Bioscan AR-2000 radio-TLC plate reader. 0.1 M citric acid (pH=4.2) was used as the elution solvent. The solution was centrifuged at 10000 rcf for 5 minutes, the supernatant removed and counted, and the product re-dispersed in 10 mM MES buffer in order to achieve purification.

The centrifugal pelleting approach to determining radiochemical yield proceeded by centrifugation of radiolabeled nanoparticles at 10000 rcf to create a pellet at the bottom of the Eppendorf tube, removal of supernatant, re-dispersion of the pellet in 10 mM IVIES and measurement of its radioactivity. Control solutions, absent of nanoparticles, were evaluated for both iTLC and pelleting to determine any gallium precipitate formation.

Serum Stability Studies:

Serum stability experiments were performed at 37° C. in a mixture of 50% fetal bovine serum (FBS, Gemini Bio-products) and 50% buffer (total volume 150 μL) on an Eppendorf thermomixer at 550 rpm. Both iTLC and size exclusion filtration analysis (100 kD filters) were completed. The size exclusion filtration analysis was conducted by placing 10 μL of 50 mM EDTA (pH 7) into the serum samples for 10 minutes, after which the samples were placed in the size exclusion filter and centrifuged at 10000 rcf for 5 minutes. Samples were washed twice, and the activity in the filter was measured and compared to the radioactivity that passes through the filter. Controls were ran absent of nanoparticles. The value reported herein is that measured by iTLC, which showed more free activity and was therefore considered to be a lower-bound estimate.

EDTA Challenge Studies:

After purification, 10 μL of 50 mM EDTA (pH 7) was added to each sample and incubated at room temperature for 3 hours. Samples were then run on iTLC as described in the radiochemical yield section. Controls were run absent of nanoparticles.

### REFERENCES

1. Hockel M, Dornhofer N. The hydra phenomenon of cancer: why tumors recur locally after microscopically complete resection. Cancer Res. 2005; 65: 2997-3002.
2. Andreou C, Kishore S A, Kircher M F. Surface-Enhanced Raman Spectroscopy: A New Modality for Cancer Imaging. J Nucl Med. 2015; 56: 1295-9.
3. Harmsen S, Bedics M A, Wall M A, Huang R, Detty M R, Kircher M F. Rational design of a chalcogenopyrylium-based surface-enhanced resonance Raman scattering nanoprobe with attomolar sensitivity. Nat Commun. 2015; 6: 6570.
4. Harmsen S, Huang R, Wall M A, Karabeber H, Samii J M, Spaliviero M, et al. Surface-enhanced resonance Raman scattering nanostars for high-precision cancer imaging. Science translational medicine. 2015; 7: 271ra7.
5. Huang R, Harmsen S, Samii J M, Karabeber H, Pitter K, Holland E C, et al. High Precision Imaging of Microscopic Spread of Glioblastoma with a Targeted Ultrasensitive SE(R)RS Molecular Imaging Probe. Theranostics. 2016; 6: 1075-84.
6. Karabeber H, Huang R, Iacono P, Samii J M, Pitter K, Holland E C, et al. Guiding brain tumor resection using surface-enhanced Raman scattering nanoparticles and a hand-held Raman scanner. ACS Nano. 2014; 8: 9755-66.
7. Kircher M F. How can we apply the use of surface-enhanced Raman scattering nanoparticles in tumor imaging? Nanomedicine (Lond). 2017; 12: 171-4.
8. Nayak T R, Andreou C, Oseledchyk A, Marcus W D, Wong H C, Massague J, et al. Tissue factor-specific ultra-bright SE(R)RS nanostars for Raman detection of pulmonary micrometastases. Nanoscale. 2017; 9: 1110-9.
9. Oseledchyk A, Andreou C, Wall M A, Kircher M F. Folate-Targeted Surface-Enhanced Resonance Raman Scattering Nanoprobe Ratiometry for Detection of Microscopic Ovarian Cancer. ACS Nano. 2017; 11: 1488-97.
10. Spaliviero M, Harmsen S, Huang R, Wall M A, Andreou C, Eastham J A, et al. Detection of Lymph Node Metastases with SE(R)RS Nanoparticles. Molecular imaging and biology: MIB: the official publication of the Academy of Molecular Imaging. 2016.
11. Wall M A, Harmsen S, Pal S, Zhang L, Arianna G, Lombardi J R, et al. Surfactant-Free Shape Control of Gold Nanoparticles Enabled by Unified Theoretical Framework of Nanocrystal Synthesis. Adv Mater. 2017; 29.
12. Harmsen S, Wall M A, Huang R, Kircher M F. Cancer imaging using surface-enhanced resonance Raman scattering nanoparticles. Nat Protoc. 2017; 12: 1400-14.
13. Kircher M F, Josephson L, Weissleder R. Ratio imaging of enzyme activity using dual wavelength optical reporters. Mol Imaging. 2002; 1: 89-95.
14. Kircher M F, de la Zerda A, Jokerst J V, Zavaleta C L, Kempen P J, Mittra E, et al. A brain tumor molecular imaging strategy using a new triple-modality MRI-photoacoustic-Raman nanoparticle. Nature medicine. 2012; 18: 829-34.
15. Thakor A S, Luong R, Paulmurugan R, Lin F I, Kempen P, Zavaleta C, et al. The fate and toxicity of Raman-active silica-gold nanoparticles in mice. Science translational medicine. 2011; 3: 79ra33.
16. Andreou C, Neuschmelting V, Tschaharganeh D F, Huang C H, Oseledchyk A, Iacono P, et al. Imaging of Liver Tumors Using Surface-Enhanced Raman Scattering Nanoparticles. ACS Nano. 2016.
17. Andreou C, Pal S, Rotter L, Yang J, Kircher M F. Molecular Imaging in Nanotechnology and Theranostics. Mol Imaging Biol. 2017; 19: 363-72.
18. Gambhir S S. Molecular imaging of cancer with positron emission tomography. Nat Rev Cancer. 2002; 2: 683-93.
19. Zavaleta C L, Hartman K B, Miao Z, James M L, Kempen P, Thakor A S, et al. Preclinical evaluation of Raman nanoparticle biodistribution for their potential use in clinical endoscopy imaging. Small. 2011; 7: 2232-40.
20. Shaffer™, Harmsen S, Khwaj a E, Kircher M F, Drain C M, Grimm J. Stable Radiolabeling of Sulfur-Functionalized Silica Nanoparticles with Copper-64. Nano Letters. 2016; 16: 5601-4.
21. Goel S, Chen F, Ehlerding E B, Cai W. Intrinsically radiolabeled nanoparticles: an emerging paradigm. Small. 2014; 10: 3825-30.
22. Phillips W T, Goins B A, Bao A. Radioactive liposomes. Wiley Interdisciplinary Reviews: Nanomedicine and Nanobiotechnology. 2009; 1: 69-83.
23. Boswell Calif., Sun X, Niu W, Weisman G R, Wong E H, Rheingold Ala., et al. Comparative in vivo Stability of Copper-64-Labeled Cross-Bridged and Conventional Tetraazamacrocyclic Complexes. Journal of Medicinal Chemistry. 2004; 47: 1465-74.
24. Zhou M, Zhang R, Huang M, Lu W, Song S, Melancon M P, et al. A Chelator-Free Multifunctional [64Cu]CuS Nanoparticle Platform for Simultaneous Micro-PET/CT Imaging and Photothermal Ablation Therapy. Journal of the American Chemical Society. 2010; 132: 15351-8.
25. Sun Y, Yu M, Liang S, Zhang Y, Li C, Mou T, et al. Fluorine-18 labeled rare-earth nanoparticles for positron emission tomography (PET) imaging of sentinel lymph node. Biomaterials. 2011; 32: 2999-3007.
26. Lovell J F, Jin C S, Huynh E, Jin H, Kim C, Rubinstein J L, et al. Porphysome nanovesicles generated by porphyrin bilayers for use as multimodal biophotonic contrast agents. Nat Mater. 2011; 10: 324-32.
27. Sun Y, Liu Q, Peng J, Feng W, Zhang Y, Yang P, et al. Radioisotope post-labeling upconversion nanophosphors for in vivo quantitative tracking. Biomaterials. 2013; 34: 2289-95.
28. Boros E, Bowen A M, Josephson L, Vasdev N, Holland J P. Chelate-free metal ion binding and heat-induced radiolabeling of iron oxide nanoparticles. Chemical Science. 2015; 6: 225-36.
29. Hoffman D, Sun M, Yang L, McDonagh P R, Corwin F, Sundaresan G, et al. Intrinsically radiolabelled [(59)Fe]-SPIONs for dual MRI/radionuclide detection. American Journal of Nuclear Medicine and Molecular Imaging. 2014; 4: 548-60.
30. Shaffer™, Wall M A, Harmsen S, Longo V A, Drain C M, Kircher M F, et al. Silica nanoparticles as substrates for chelator-free labeling of oxophilic radioisotopes. Nano Lett. 2015; 15: 864-8.
31. Chen F, Hong H, Goel S, Graves S A, Orbay H, Ehlerding E B, et al. In vivo Tumor Vasculature Targeting of CuS@MSN Based Theranostic Nanomedicine. ACS Nano. 2015; 9: 3926-34.
32. Kinraide T B, Yermiyahu U. A scale of metal ion binding strengths correlating with ionic charge, Pauling electronegativity, toxicity, and other physiological effects. Journal of inorganic biochemistry. 2007; 101: 1201-13.
33. Decuzzi P, Godin B, Tanaka T, Lee S Y, Chiappini C, Liu X, et al. Size and shape effects in the biodistribution of intravascularly injected particles. Journal of controlled release: official journal of the Controlled Release Society. 2010; 141: 320-7.
34. Le V S.-68Ga Generator Integrated System: Elution—Purification—Concentration Integration. 2013:-75.
35. Brinker C J. Hydrolysis and condensation of silicates—effects on structure. Journal of Non-Crystalline Solids. 1988; 100: 31-50.
36. Carcouet C C, van de Put M W, Mezari B, Magusin P C, Laven J, Bomans P H, et al. Nucleation and growth of monodisperse silica nanoparticles. Nano letters. 2014; 14: 1433-8.
37. Keefer K D, Schaefer D W. Growth of fractally rough colloids. Physical review letters. 1986; 56: 2376-9.
38. Sintes T, Toral R, Chakrabarti A. Fractal structure of silica colloids revisited. Journal of Physics a-Mathematical and General. 1996; 29: 533-40.
39. Wijnen P, Beelen T P M, Dehaan J W, Rummens C P J, Vandeven L J M, Vansanten R A. Silica-gel dissolution in aqueous alkali-metal hydroxides studied by SI-29-NMR. Journal of Non-Crystalline Solids. 1989; 109: 85-94.
40. Velikyan I. 68Ga-Based Radiopharmaceuticals: Production and Application Relationship. Molecules. 2015; 20: 12913.
41. Uren R F, Howman-Giles R B, Chung D, Thompson J F. Role of lymphoscintigraphy for selective sentinel lymphadenectomy. Cancer Treat Res. 2005; 127: 15-38.
42. Iacono P, Karabeber H, Kircher M F. A “schizophotonic” all-in-one nanoparticle coating for multiplexed SE(R)RS biomedical imaging. Angew Chem Int Ed Engl. 2014; 53: 11756-61.
43. Gill R K, Mitchell G S, Cherry S R. Computed Cerenkov luminescence yields for radionuclides used in biology and medicine. Physics in medicine and biology. 2015; 60: 4263-80.
44. Shaffer T_{M}, Pratt E C, Grimm J. Utilizing the power of Cerenkov light with nanotechnology. Nat Nano. 2017; 12: 106-17.
45. Hama Y, Koyama Y, Urano Y, Choyke P L, Kobayashi H. Two-color lymphatic mapping using Ig-conjugated near infrared optical probes. J Invest Dermatol. 2007; 127: 2351-6.
46. Zhang F, Niu G, Lu G, Chen X. Preclinical lymphatic imaging. Mol Imaging Biol. 2011; 13: 599-612.
47. Kumar B, Miller T R, Siegel B A, Mathias C J, Markham J, Ehrhardt G J, et al. Positron tomographic imaging of the liver: 68 Ga iron hydroxide colloid. AJR Am J Roentgenol. 1981; 136: 685-90.
48. Na H B, Song I C, Hyeon T. Inorganic Nanoparticles for MRI Contrast Agents. Advanced Materials. 2009; 21: 2133-48.
49. Andreou C, Neuschmelting V, Tschaharganeh D F, Huang C H, Oseledchyk A, Iacono P, et al. Imaging of Liver Tumors Using Surface-Enhanced Raman Scattering Nanoparticles. ACS nano. 2016; 10: 5015-26.

What is claimed is:

1. A method of preparing medical isotope labeled nanoparticles, the method comprising steps of:
providing a reaction mixture comprising or consisting of:
(a) nanoparticles comprising a metal or metal alloy core, a plurality of capping agent entities associated on the core, an outer silica encapsulant layer, and a plurality of SE(R)RS-active agent dopant entities; and
(b) medical isotopes,
(c) soft cations,
wherein the reaction mixture is substantially free of chelator; and
maintaining the reaction mixture under conditions and for a time sufficient for the medical isotopes to bind with the nanoparticles, thereby forming medical isotope labeled nanoparticles.

2. The method of claim 1, further comprising a step of isolating the labeled nanoparticles.

3. The method of claim 2, wherein the step of isolating the medical isotope labeled nanoparticles comprises centrifuging the reaction mixture.

4. The method of claim 2, wherein the step of isolating the medical isotope labeled nanoparticles comprises filtrating the reaction mixture.

5. The method of any one of the preceding claims, further comprising dispersing the isolated medical isotope labeled nanoparticles in an infusion fluid.

6. The method of any one of the preceding claims, wherein the step of maintaining the reaction mixture under conditions comprises heating the reaction mixture to a temperature of equal to or greater than 25° C.

7. The method of any one of the preceding claims, wherein the step of maintaining the reaction mixture under conditions comprises heating the reaction mixture to a temperature of between 45° C. and 80° C.

8. The method of any one of the preceding claims, wherein the step of maintaining the reaction mixture under conditions comprises heating the reaction mixture to a temperature of equal to or greater than 95° C.

9. The method of any one of the preceding claims, wherein the time is between 5 and 120 minutes.

10. The method of any one of the preceding claims, further comprising administering the medical isotope labeled nanoparticles to a subject in vivo.

11. The method of any one of the preceding claims, wherein integrity of the medical isotope labeled nanoparticles is not affected by the steps of providing the reaction mixture and maintaining the reaction mixture.

12. The method of any one of the preceding claims, wherein a binding between the nanoparticles and the medical isotope is covalent.

13. The method of any one of claims 1-11, wherein a binding between the nanoparticles and the medical isotope is non-covalent.

14. The method of claim 13, wherein the binding between the nanoparticles and the medical isotope is via chelate bonds.

15. The method of any one of the preceding claims, wherein the nanoparticles have a longest dimension between 2-1000 nm.

16. A kit for production of medical isotope labeled nanoparticle agents for imaging and/or therapeutics, comprising:
nanoparticles comprising a metal or metal alloy core, a plurality of capping agent entities associated on the core, an outer silica encapsulant layer, and a plurality of SE(R)RS-active agent dopant entities,
wherein the nanoparticles are characterized in that, when exposed to an elevated temperature, the nanoparticles bind a plurality of medical isotopes in the presence of soft cations.

17. The kit of claim 16, further comprising reagents for combining the nanoparticles with the plurality of medical isotopes.

18. The kit of claim 17, wherein the reagents comprise soft cations.

19. The kit of any one of claims 16-18, further comprising a buffer and/or an infusion fluid.

20. The kit of any one of claims 16-19, further comprising a device for administering the medical isotope labeled nanoparticle agent to a subject.

21. The kit of claim 20, wherein the device is a syringe.

22. The kit of any one of claims 16-21, wherein the nanoparticles have a longest dimension between 2-1000 nm.

23. The kit of any one of claims 16-22, wherein the nanoparticles bind the plurality of medical isotopes via covalent bonds.

24. The kit of any one of claims 16-22, wherein the nanoparticles bind the plurality of medical isotopes via non-covalent bonds.

25. The kit of claim 24, wherein the nanoparticles bind the plurality of medical isotopes via chelate bonds.

26. A medical isotope labeled nanoparticle agent, comprising:
a nanoparticle comprising a metal or metal alloy core, a plurality of capping agent entities associated on the core, an outer silica encapsulant layer, and a plurality of SE(R)RS-active agent dopant entities, the nanoparticle bound to a medical isotope,
wherein the nanoparticle agent is characterized in that it is stable in vivo for at least 3 hours.

27. The medical isotope labeled nanoparticle agent of claim 26, wherein a specific activity of the nanoparticle is no less than 1 Ci/μmol.

28. The medical isotope labeled nanoparticle agent of claims 26-27, wherein the plurality of the SE(R)RS-active agent dopant entities is present at sufficiently high density and in sufficient proximity to a surface of the metal or metal alloy that the particle displays ultrahigh Raman sensitivity.

29. The medical isotope labeled nanoparticle agent of claims 26-28, wherein the nanoparticle agent is characterized in that it localizes in one or more regions selected from the list consisting of liver, spleen, tumor, lymph node, inflammation, and infections.

30. The medical isotope labeled nanoparticle agent of any one of claims 26-29, wherein the nanoparticle agent is characterized in that it comprises at least one targeting moiety/agent.

31. The medical isotope labeled nanoparticle agent of claim 30, wherein the targeting moiety comprises at least one agent selected from the list consisting of antibodies, peptides, aptamers, small molecular targeting agent entities, and any combination thereof.

32. The medical isotope labeled nanoparticle agent of any one of claims 26-31, wherein the nanoparticle agent is characterized in that it comprises at least one click reagent.

33. The medical isotope labeled nanoparticle agent of claim 32, wherein the click reagent comprises at least one agent selected from the list consisting of alkynes, azides, cyclooctynes (e.g., (sulfo-)dibenzocyclooctynes, (1R,8 S,9s)-Bicyclo[6.1.0]non-4-yn-9-yls (BCN), (E)-Cyclooctynes, TCO, etc.), isonitriles, ketones, nitrones, oximes, quadricyclanes, and tetrazines.

34. The medical isotope labeled nanoparticle agent of any one of claims 26-33, wherein the nanoparticle agent is characterized in that the labeled nanoparticle is used for at least one purpose selected from the group consisting of preclinical research, biomedical imaging, therapy, intraoperative imaging, and surgery preparation and/or planning.

35. The medical isotope labeled nanoparticle agent of any one of claims 26-34, wherein the nanoparticle agent is characterized in that the nanoparticle is bound to the medical isotope via covalent bonds.

36. The medical isotope labeled nanoparticle agent of any one of claims 26-34, wherein the nanoparticle agent is characterized in that the nanoparticle is bound to the medical isotope via non-covalent bonds.

37. The medical isotope labeled nanoparticle agent of any one of claims 26-34, wherein the nanoparticle agent is characterized in that the nanoparticle is bound to the medical isotope via chelate bonds.
